(19) United States
(12) Patent Application Publication
(10) Pub. No.: US 2019/0314069 A1
(43) Pub. Date: Oct. 17, 2019

(54) Polyaxial Bone Fixation System

(71) Applicant: Aristotech Holding GmbH, Berlin (DE)

(72) Inventor: Emmanuel Anapliotis, Berlin (DE)

(73) Assignee: Aristotech Holding GmbH, Berlin (DE)

(21) Appl. No.: 16/309,027

(22) PCT Filed: Jul. 18, 2016
(86) PCT No.: PCT/DE2016/000286

§ 371 Date: Dec. 11, 2018

(51) Int. Cl. A61B 17/80; A61B 17/86

(52) U.S. Cl. CPC A61B 17/8052; A61B 2017/00946; A61B 17/8605

(57) ABSTRACT

A polyaxial bone fixation system has a bone screw with a head having an outer thread and a shaft. A bone plate has an upper face and a lower face and a through-bore for receiving the bone screw. The bone screw is introduced into the through-bore at a selectable angle and can be fixed in the through-bore at the selected angle. Material openings extend around the through-bore such that between the through-bore and each material recess opening, a bridge is formed which runs continuously from the upper face to the lower face. The bridge forms a protuberance which protrudes into the through-bore and runs in the longitudinal direction of the through-bore. The radius of the through-bore is reduced in the region of the protuberance. The bridge can plastically deform when the bone screw is screwed into the through-bore.

The invention relates to a polyaxially screwable bone plate and a bone fixation system which comprises a bone screw with a bone screw head having an outer thread and a bone screw shaft as well as a bone plate with an upper face and a lower face, wherein the bone plate has at least one through-bore reaching from the upper face to the lower face for receiving the bone screw, and the bone screw may be introduced into the through-bore at a selectable angle and can be fixed in the through-bore at the selected angle with angular stability.

From the prior art, such bone fixation systems are known for treating fractures, in which the bone screw for fixation of the bone plate on the bone is inserted into the through-bore under polyaxial angular conditions and can be set therein with angular stability. Setting of the angle between the bone screw and bone plate in these systems occurs while the bone screw is screwed in the bone.

A polyaxial fixation system is known from DE 43 43 117 C2 in which both the thread of the bone screw head and the thread in the through-bore can have thread segments separated from one another, whose ranges of separation favor the penetration of a mating thread at various screwing angles.

EP 1 935 359 B1 discloses a bone plate that has at least two through-bores. The through-bores are offset from one another and intersect one another such that the lines of intersection and/or the surfaces of intersection are in the direction of the plate thickness, wherein the lines of intersection interact with the thread of the bone screw head. During the screwing, plastic deformation takes place between the bone screw head of the screwed bone screw and the intersection lines/intersection surfaces, which results in a nonpositive/positive connection between the plate and the bone screw.

The prior art further describes solutions in which the through-bores are conical or double-coned.

DE 10 2010 048 052 A1 discloses a bone plate in which the through-bore is double-coned and has a double-coned thread or a double-coned thread star in the region of the smallest diameter.

The bone screw can be provided with a clamping sleeve having a spherical portion of an outer thread, by means of which a polyaxial alignment of the bone screw occurs in the region of the thread of the through-bore.

EP 1 919 385 B1 describes a polyaxial bone fixation arrangement in which, in the through-bore of a bone plate, a plurality of projecting ribs are arranged which are pliable and with which the thread of a bone screw interacts.

The object of the invention is to provide an improved polyaxial bone fixation system for fixation of a bone plate with angular stability having at least one bone screw, which in addition is simple to produce.

So as to accomplish the object, a polyaxial bone fixation system is provided having a bone screw with a bone screw head having an outer thread and a bone screw shaft as well as a bone plate with an upper face and a lower face, in which, in the bone plate, at least one through-bore is set which extends from the upper face to the lower face for receiving the bone screw, wherein the bone screw may be introduced into the through-bore at a polyaxial angle and in the selected angle can be fixed or can be set with angular stability.

Around a through-bore close to its circumferential edge, several material recess openings reaching from the upper face to the lower face of the bone plate are set up in such a way that a web is formed between the through-bore and each material recess opening and extends all the way from the upper face to the lower face of the bone plate. It is essential that each web has a bulge which projects into the through-bore and runs in the longitudinal direction of the through-bore. In addition, each web is configured such that it is plastically deformed during screwing of the bone screw in a polyaxial angle with the through-bore.

The term “plastic deformability,” in contrast to elastic deformability, indicates permanent shaping of the material. “Through-bore” indicates a bore that goes from the upper face to the lower face of the bone plate and is configured to receive a bone screw under polyaxial conditions so that the bone screw can be set with angular stability in the through-bore at an angle deviating from the longitudinal axis of the through-bore. The through-bore can be cylindrical, conical, or double-coned in the longitudinal direction.

The angle describing polyaxiality indicates the angle between the longitudinal axis of the through-bore and the longitudinal axis of the bone screw introduced into the bone plate when the bone screw is set obliquely to the longitudinal axis of the through-bore in the through-bore.

The fixation region is a region in the through-bore in which the bone screw head is set in the through-bore.

The wall of the web on the through-bore side forms a partial surface of the inner surface of the through-bore. In one advantageous embodiment, a through-bore on at least one segment of the circumferential inner surface of the inner surface, preferably on the entire circumferential inner surface, is provided with a continuous inner thread with which the outer thread on the bone screw head of the bone screw interacts. According to the definition, the term “inner surface of the through-bore” comprises the walls of the webs projecting into the through-bore, so that the inner thread then also extends over the walls of the webs.

The “material recess opening” is a recess, space, or bore-hole that extends from the upper face to the lower face of the bone plate, which is introduced at a slight distance from the circumferential edge of the through-bore but does not intersect the through-bore, so that a web remains between a material recess opening and a through-bore which runs continuously from the upper face to the lower face of the bone plate and delimits the through-bore from the material recess opening.

In a preferred embodiment, the longitudinal axes of the material recess openings are aligned parallel to the longitudinal axis of the through-bore. The cross-sectional shape of a material recess opening is preferably circular or ellipsoid, but can also have other cross-sectional shapes such as rectangular or polygonal. The embodiment of a material recess opening can differ from the embodiment of an additional material recess opening. Likewise, the cross-sectional shape can vary over the length of a material recess opening.

On the through-bore side, the web is configured as a bulge projecting into the through-bore, so that in the region of the bulge the radius or the edge distance to the midpoint of the through-bore is decreased or shortened. Radius and edge distance are treated as synonyms in the further context.

A material recess opening preferably has a circular cross-section. In alternative embodiments, the cross-section of the material recess opening can likewise possess a polygonal geometry such as a triangular or rectangular cross-sectional shape. In other embodiments, the material recess opening can have ellipsoid or even geometrically irregular cross-sectional forms.

In a preferred embodiment, the cross-sectional shape and/or its external dimensions of the material recess opening over its course from the upper face to the lower face of the bone plate remain constant. In alternative designs the material recess opening can be conical or in other cases also pyramidally tapered (in the shape of a truncated pyramid). The maximal cross-section width of a material recess opening is basically smaller than the maximal cross-sectional width of the through-bore by a multiple thereof.

With the arrangement of one or more material recess openings in a region of the through-bore close to the edge, a deformation zone is produced there that permits a plastic deformation of the web into a cavity.

Due to plastic deformability of one or more webs, for screwing the bone plate in or on the bone, the shape of the fixation region can be adapted to the required angular position of the bone screw by pressing one or more webs due to the force effect from screwing the bone screw into the through-bore at least in a partial region in the direction of the material recess opening located behind a web.

If now the bone screw is inserted obliquely into the through-bore and screwed therein, with plastic deformation of the web, a nonpositive/positive connection is created between the bone screw and the fixation region in the through-bore, which ensures the angular stability of the screwing process. Preferably the bone screw can be set in the through-bore at an angle of 0° to 15° with angular stability.

The degree of plastic deformability of a web is determined in particular by its local wall thickness and/or by the hardness of the bone plate material. In one particular embodiment, the wall thickness of the web increases over the length of the through-bore toward one side of the bone plate. In addition, alternating regions with thicker and/or thinner wall thicknesses can be set up in the longitudinal direction of the through-bore.

Due to a bulge projecting into the through-bore, the radius of the through-bore is reduced in comparison with the circumferential regions behind which no material recess opening is set.

In one embodiment, the bulge projecting into the through-bore can be formed when the web has a convex bend facing in the radial direction toward the longitudinal axis of the through-bore and extending over the entire length of the through-bore.

In an alternative embodiment, the bulge is formed by a material thickening in the web facing in the direction of the longitudinal axis of the through-bore.

In a preferred embodiment, the circular diameter between the innermost points of at least two webs is equal to the largest core diameter of the bone screw head.

In a further embodiment some, advantageously all material recess openings are arranged on a common circular path whose midpoint lies in the longitudinal axis of the through-bore. The material recess openings that are arranged on the common circular path are set in one embodiment uniformly separated from one another

In another advantageous embodiment, at least two of the material recess openings are arranged axis-symmetrically with respect to the longitudinal axis of the through-bore.

A bone plate according to the invention of the bone fixation system according to the invention is preferably produced by means of a production method which comprises at least the following production steps.
boring or milling a through-bore from the upper face to the lower face through the bone plate;
determining the location and the diameter of a material recess opening close to the circumferential edge of the through-bore without the material recess opening intersecting the through-bore;
boring or milling the material recess opening through the bone plate with formation of a thin-walled web between the material recess opening and the through-bore, wherein during the boring or milling process the thin-walled web is deformed in such a way that its wall on the through-bore side projects into the through-bore with formation of a bulge.

If the circumferential inner surface or a circumferential inner surface section of the inner surface of the through-bore is equipped with a thread, the thread is cut after boring or milling of a through-bore through the bone plate into the circumferential inner surface or the circumferential inner surface section before the recess openings passing from the upper face to the lower face through the bone plate close to the circumferential edge of the through-bore are produced.

This simple production method makes possible in particular the production of small-part polyaxially screwable bone fixation systems that can be used in pediatric surgery or also in veterinary medicine for surgical fracture treatment of small animals.

Below, the polyaxial bone fixation system according to the invention is explained in more detail with reference to an exemplary embodiment. The figures show:

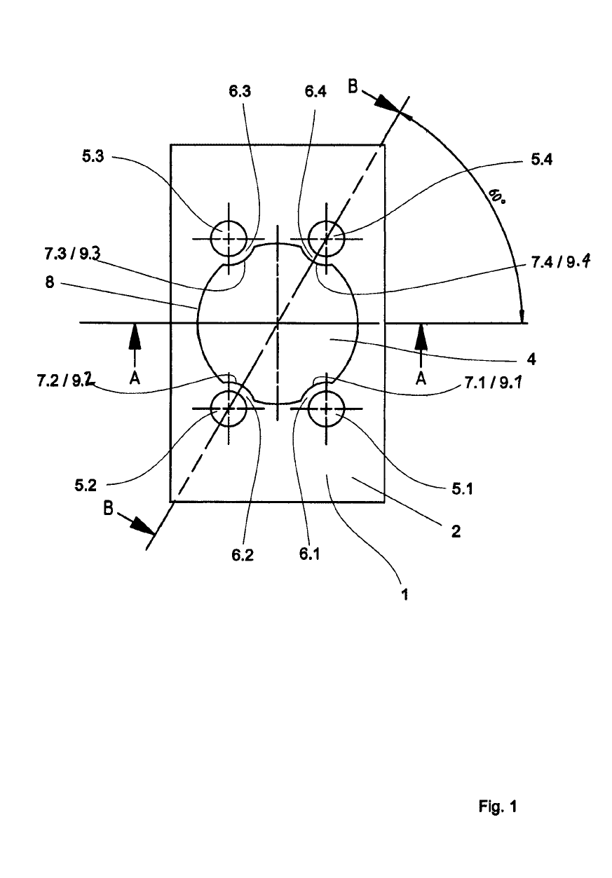

FIG. 1: a two dimensional representation of a bone plate section with through-bore and four material recess openings arranged around it;

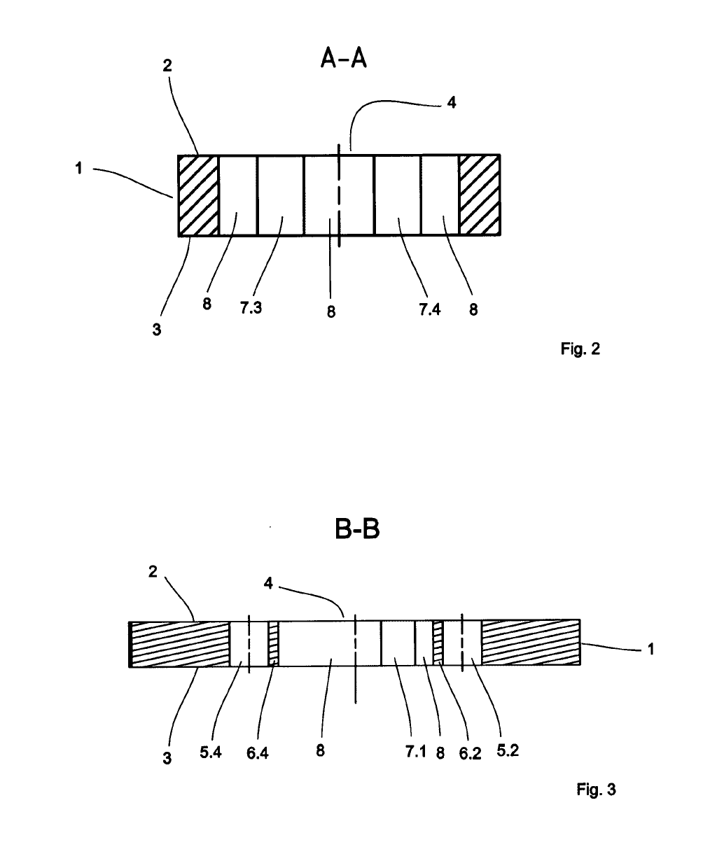

FIG. 2: view of the bone plate section according to FIG. 1 in section A-A,

FIG. 3: view of the bone plate section according to FIG. 1 in section B-B and

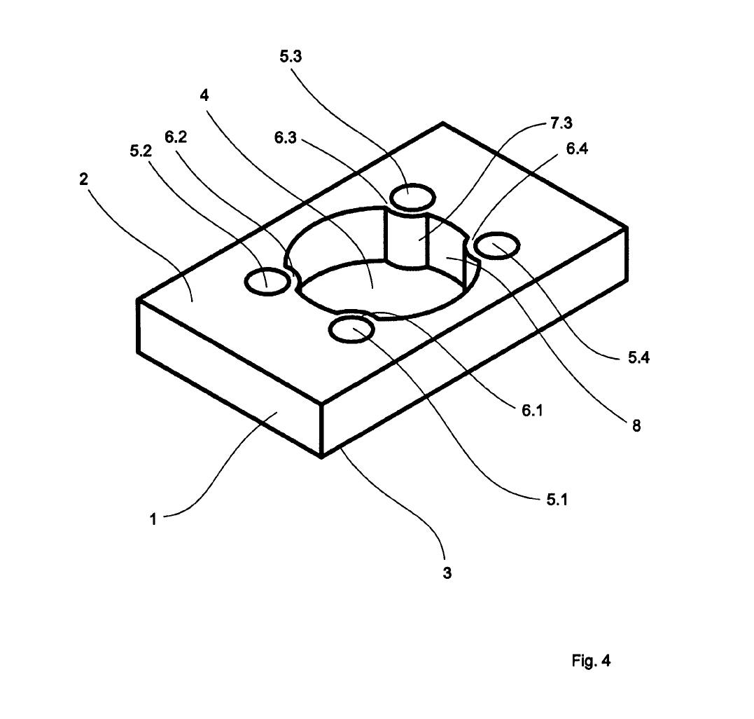

FIG. 4: a three-dimensional representation of a bone plate section according to FIG. 1 with a through-bore and four material recess openings arranged around it.

FIGS. 1 to 4 show a bone plate section of a bone plate 1 with a through-bore 4 passing from the upper face 2 to the lower face 3 and four material recess openings 5.1, 5.2, 5.3, and 5.4 arranged around the through-bore 4, wherein each material recess opening 5.1, 5.2, 5.3, and 5.4 runs continuously from the upper face 2 to the lower face 3 of the bone plate 1 and is set close to the circumferential edge of the through-bore 4. It is essential that none of the material recess openings 5.1, 5.2, 5.3, and 5.4 intersects the through-bore 4.

Between each material recess opening 5.1, 5.2, 5.3, and 5.4 and the through-bore 4, there is a thin-walled web 6.1, 6.2, 6.3, and 6.4 which projects into the through-bore 4 and in each case forms a bulge 9.1, 9.2, 9.3, and 9.4, wherein in the region of the bulges 9.1, 9.2, 9.3, and 9.4 projecting into the through-bore, in each case the radius of the through-bore 4 is reduced.

The specific walls 7.1, 7.2, 7.3, and 7.4 of the webs 6.1, 6.2, 6.3, and 6.4 each form a section of the circumferential inner surface 8 of the through-bore 4. Each wall 7.1, 7.2, 7.3, and 7.4 has convex curve facing in the radial direction to the longitudinal axis of the through-bore 4 and extending over the entire length of the through-bore 4.

All four material recess openings 5.1, 5.2, 5.3, and 5.4 are circular in cross section and cylindrical in the longitudinal direction and much smaller than the through-bore. Their longitudinal axes run parallel to the longitudinal axis of the through-bore 4, which is orthogonally aligned with the surface 2 of the bone plate section. In addition, in each case two material recess openings 5.1 and 5.3 or 5.2 and 5.4 are arranged axis-symmetrically with respect to the longitudinal axis of the through-bore 4. In addition, the four material recess openings 5.1, 5.2, 5.3, and 5.4 are set with their midpoint axes on a common circular path whose midpoint lies in the longitudinal axis of the through-bore.

Each material recess opening 5.1, 5.2, 5.3, and 5.4 forms with their webs 6.1, 6.2, 6.3, and 6.4 a deformation zone in the edge region of the through-bore 4, so that depending on the specific insertion angle of the bone screw, one or more of the webs 6.1, 6.2, 6.3, and 6.4 can be deformed at least in partial regions, by which the required angular stability of the screwed-in bone screw is achieved.

1. A polyaxial bone fixation system comprising:
a bone screw with a bone screw head having an outer thread and a bone screw shaft as well as a bone plate with a upper face and a lower face, wherein in the bone plate at least one through-bore passing from the upper face to the lower face is set up to receive the bone screw, wherein the bone screw can be introduced into the through-bore at a selectable angle and can be fixed in the through-bore in the selected angle with angular stability,
wherein around the through-bore, several material recess openings reaching from the upper face to the lower face of the bone plate are set such that between the through-bore and each material recess opening in each case a web is formed which runs continuously from the upper face to the lower face of the bone plate, and
wherein the web forms a bulge projecting into the through-bore and running in the longitudinal direction of the through-bore, and in the region of the bulge projecting into the through-bore, the radius of the through-bore is reduced, and wherein the web is plastically deformable upon screwing of the bone screw into the through-bore.

2. The polyaxial bone fixation system according to claim 1, wherein
the through-bore has a continuous inner thread on at least one circumferential inner surface section of the inner surfaces.

3. The polyaxial bone fixation system according to claim 1, wherein
the longitudinal axes of the material recess openings are aligned parallel to the longitudinal axis of the through-bore.

4. The polyaxial bone fixation system according to claim 1, wherein
the material recess openings are arranged on a common circular path about the longitudinal axis of the through-bore.

5. The polyaxial bone fixation system according to claim 1, wherein
the material recess openings are arranged axis-symmetrically to the longitudinal axis of the through-bore.

6. The polyaxial bone fixation system according to claim 1, wherein
the material recess openings are arranged uniformly separated from one another.

7. The polyaxial bone fixation system according to claim 1, wherein
a material recess opening has a circular, ellipsoid, rectangular, or polygonal cross section.

8. The polyaxial bone fixation system according to claim 1, wherein
the polyaxial angle between the longitudinal axis of the through-bore and the longitudinal axis of a bone screw is 0° to 15°.

9. The polyaxial bone fixation system according to claim 1, wherein
the web has a convex curve facing in the radial direction toward the longitudinal axis of the through-bore and extending over the entire length of the through-bore.

10. A bone plate polyaxially screwable with a bone having an upper face and a lower face, the bone plate comprising:
at least one through-bore passing from the upper face to the lower face and being set for receiving a bone screw, into which a bone screw can be introduced at a selectable angle and can be fixed at the selected angle with angular stability in the through-bore
wherein around the through-bore, several material recess openings reaching from the upper face to the lower face of the bone plate are set in such a way that between the through-bore and each material recess opening, in each case a web is formed, which runs continuously from the upper face to the lower face of the bone plate, and
wherein the web forms a bulge projecting into the through-bore and running in the longitudinal direction of the through-bore, and in the region of the bulge projecting into the through-bore, the radius of the through-bore is reduced, and wherein the web is plastically deformable upon screwing of the bone screw into the through-bore.

11. The polyaxial bone fixation system according to claim 2, wherein
the longitudinal axes of the material recess openings are aligned parallel to the longitudinal axis of the through-bore.

12. The polyaxial bone fixation system according to claim 2, wherein
the material recess openings are arranged on a common circular path about the longitudinal axis of the through-bore.

13. The polyaxial bone fixation system according to claim 3, wherein
the material recess openings are arranged on a common circular path about the longitudinal axis of the through-bore.

14. The polyaxial bone fixation system according to claim 2, wherein
the material recess openings are arranged axis-symmetrically to the longitudinal axis of the through-bore.

15. The polyaxial bone fixation system according to claim 3, wherein
the material recess openings are arranged axis-symmetrically to the longitudinal axis of the through-bore.

16. The polyaxial bone fixation system according to claim 4, wherein
the material recess openings are arranged axis-symmetrically to the longitudinal axis of the through-bore.

17. The polyaxial bone fixation system according to claim 2, wherein
the material recess openings are arranged uniformly separated from one another.

18. The polyaxial bone fixation system according to claim 3, wherein
the material recess openings are arranged uniformly separated from one another.

19. The polyaxial bone fixation system according to claim 4, wherein
the material recess openings are arranged uniformly separated from one another.

20. The polyaxial bone fixation system according to claim 5, wherein
the material recess openings are arranged uniformly separated from one another.
